SEQUENCE LISTING (2 sequences)

SEQ ID NO: 1 - protein, 425 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 2 - protein, 28 aa, Homo sapiens
RSGPPGLQGRLQRLLQASGNHAAGILTM

(19) United States
(12) Patent Application Publication
(10) Pub. No.: US 2018/0044393 A1
(43) Pub. Date: Feb. 15, 2018

(54) OREXIN-B POLYPEPTIDES AND USES THEREOF

(71) Applicants: INSERM (INSTITUT NATIONAL DE LA SANTÉ ET DE LA RECHERCHE MÉDICALE), Paris (FR); UNIVERSITÉ PARIS DIDEROT - PARIS 7, Paris (FR); INSTITUT RÉGIONAL DU CANCER DE MONTPELLIER, Montpellier (FR); UNIVERSITÉ DE MONTPELLIER, Montpellier (FR)

(72) Inventors: Alain COUVINEAU, Paris Cedex 18 (FR); Thierry VOISIN, Paris Cedex 18 (FR)

(21) Appl. No.: 15/551,334

(22) PCT Filed: Feb. 18, 2016
(86) PCT No.: PCT/EP2016/053476

§ 371 Date: Aug. 16, 2017

(30) Foreign Application Priority Data
Feb. 19, 2015 (EP) 15305247.7

(51) Int. Cl. C07K 14/575

(52) U.S. Cl. CPC C07K 14/575; C07K 2319/30; A61K 38/00

(57) ABSTRACT

The present invention relates to Orexin-B polypeptides and uses thereof, in particular for the treatment of cancer. In particular, the polypeptide of the present invention comprises the amino acid sequence ranging from the amino acid residue at position 6 to the amino acid residue at position 28 in SEQ ID NO:2 wherein at least one amino acid residue position 6, 7, 8, 9, 10, 12, 13, 14, 19, 21 or 23 is substituted and the amino acid residues at position 11; 15; 16; 17; 18; 20; 22; 24; 25; 26; 27; and 28 are not deleted or substituted.

### FIELD OF THE PRESENT INVENTION

The present invention relates to Orexin-B polypeptides and uses thereof, in particular for the treatment of cancer.

### BACKGROUND OF THE PRESENT INVENTION

Orexins A and B (also known as hypocretins 1 and 2) are hypothalamic 33-aminoacid and 28-aminoacid neuropeptides, respectively, which originate from prepro-orexin, a 131-residue precursor. Orexin-A (OxA) contains two intramolecular disulfide bonds between positions 6 to 12 and 7 to 14 while orexin-B (OxB) does not have any. These two peptides share the same effects, regulating sleep, wakefulness, feeding, energy homeostasis, obesity, diabetes, breathing, reward system or drug addiction (Laburthe and Voisin, 2012). Orexins trigger biological effects by interacting with 2 members of the class A G-protein coupled receptor (GPCRs) family, i.e., orexin receptor-1 (OX1R) and orexin receptor-2 (OX2R) (Thompson et al., 2014). Activation of these receptors by orexins classically induces cellular calcium transients through Gq-dependent and -independent pathways (Laburthe et al., 2010). Besides these central actions, the orexins/receptor system is also involved in peripheral effects, including cardiovascular modulation, and neuroendocrine and reproduction regulation (Xu et al., 2013). Recently, our group demonstrated that OxA and OxB, bound to OX1R, can induce massive apoptosis, resulting in the drastic reduction of cell growth in various colonic cancer cell lines, including HT-29, LoVo, Caco-2 and others (Voisin et al., 2011). An entirely novel mechanism, not related to Gq-mediated phospholipase C activation, was shown to trigger orexin-induced apoptosis (Voisin et al., 2008; El Firar et al., 2009). In fact, orexins induced the tyrosine phosphorylation of two immunoreceptor tyrosine-based motifs (ITIMs) located at the interface between transmembrane domain (TM) 2 and TM 7 of OX1R and the cytoplasm (Voisin et al., 2008). The resulting phosphorylated receptor could then recruit and activate the phosphotyrosine phosphatase, SHP-2, which is responsible for mitochondrial apoptosis, involving cytochrome c release from mitochondria to cytosol and caspase-3 and caspase-7 activation (El Firar et al., 2009). The pro-apoptotic effect of orexins has also been extended to other cancer cell lines derived from human neuroblastoma (SK-N-MC cell line) and rat pancreatic cancer (AR42J cell line) (Rouet-Benzineb et al., 2004; Voisin et al., 2006). Recent data demonstrated that OX1R is aberrantly expressed in all resected primary colorectal tumors and liver metastases tested, but is not present in normal colon tissues (Voisin et al., 2011). Moreover, injection of exogenous orexins to mice strongly reduced in vivo tumor growth and reversed the development of established tumors in mice xenografted with colon cancer cell lines such as HT-29 or LoVo, due to robust apoptosis induction (Voisin et al., 2011). Taken together, these observations suggest that the orexins/OX1R system may represent a new promising target in colorectal cancer therapy, and most probably in other cancers, including pancreatic cancers neuroblastoma, and/or prostate cancer (Alexandre et al., 2014). In this context, structure-function relationship studies of the orexins/OX1R system are essential for the development of new agonists of OX1R that may represent new therapeutic approaches.

Until now, little has been known about the structure-function relationship of the orexins/OX1R system. The determination of the 3D structure of OxB in solution by two-dimensional NMR spectroscopy revealed the presence of two α-helices encompassing residues Leu7 to Gly19 and residues Ala23 to Met28, connected by a short flexible loop (Lee et al., 1999). In addition, pharmacological tools have been developed to discriminate between OX1R and OX2R (Laburthe and Voisin, 2012; Gotter et al., 2012), including selective peptide agonists for OX2R such as [Ala11, D-Leu15] orexin-B 6-28 (Asahi et al., 2003), [Ala27] orexin-B 6-28 (Lang et al., 2004), [Prol 1] orexin-B 6-28 (Lang et al., 2004) and other selective non-peptide antagonists, including TCS-OX2-29, JNJ-10397049, EMP4 for OX2R or the non-peptide molecule antagonists SB-334867, SB-408124 and SB-674042 for OX1R (review in Gotter et al., 2012). Nevertheless, the residues of OxB and OX1R involved in apoptosis are unknown.

### SUMMARY OF THE PRESENT INVENTION

The present invention relates to Orexin-B polypeptides and uses thereof, in particular for the treatment of cancer. In particular, the present invention is defined by the claims.

### DETAILED DESCRIPTION OF THE PRESENT INVENTION

Orexins (A and B) are hypothalamic peptides interacting with two class A GPCR subtypes, OX1R and OX2R, and involved in the sleep/wake cycle. The inventors previously demonstrated that OX1R is highly expressed in colon cancer tumors and colonic cancer cell lines where orexins induce apoptosis and inhibition of tumor growth in preclinical animal models. The inventors have now explored the structure-function relationships of orexin-B (OxB) and OX1R. The contribution of all OxB residues in OxB-induced apoptosis was indeed investigated by alanine-scanning. Alanine substitution of OxB residues, L¹¹, L¹⁵, A²², G²⁴, I²⁵, L²⁶, and M²⁸, altered OxB binding affinity. Substitution of these residues and of the Q¹⁶, A¹⁷, S¹⁸, N²⁰ and T²⁷ residues inhibited apoptosis in CHO—S-OX1R cells. These results indicate that the C-terminus of OxB 1) plays an important role in the pro-apoptotic effect of the peptide; 2) interacts with some residues localized into the OX1R transmembrane domains. This study defines the structure-function relationship for OxB recognition by human OX1R and OxB/OX1R-induced apoptosis, and thus provides a rational for the development of new polypeptides which act as OX1R agonists.

As used herein, the term “OX1R” has its general meaning in the art and refers to the 7-transmembrane spanning receptor OX1R for orexins. According to the invention, OX1R promotes apoptosis in the human prancreatic cancer cell line through a mechanism which is not related to Gq-mediated phopholipase C activation and cellular calcium transients. Orexins induce indeed tyrosine phosphorylation of 2 tyrosine-based motifs in OX1R, ITIM and ITSM, resulting in the recruitment of the phosphotyrosine phosphatase SHP-2, the activation of which is responsible for mitochondrial apoptosis (Voisin T, El Firar A, Rouyer-Fessard C, Gratio V, Laburthe M. A hallmark of immunoreceptor, the tyrosine-based inhibitory motif ITIM, is present in the G protein-coupled receptor OX1R for orexins and drives apoptosis: a novel mechanism. FASEB J. 2008 June; 22(6):1993-2002; El Firar A, Voisin T, Rouyer-Fessard C, Ostuni M A, Couvineau A, Laburthe M. Discovery of a functional immunoreceptor tyrosine-based switch motif in a 7-transmembrane-spanning receptor: role in the orexin receptor OX1R-driven apoptosis. FASEB J. 2009 December; 23(12):4069-80. doi: 10.1096/J.09-131367. Epub 2009 Aug. 6.). An exemplary amino acid sequence of OX1R is shown as SEQ ID NO:1.

Orexin receptor-1 OX1R_homo sapiens
(SEQ ID NO: 1)
MEPSATPGAQMGVPPGSREPSPVPPDYEDEFLRYLWRDYLYPKQYE
WVLIAAYVAVFVVALVGNTLVCLAVWRNHHMRTVTNYFIVNLSLAD
VLVTAICLPASLLVDITESWLFGHALCKVIPYLQAVSVSVAVLTLS
FIALDRWYAICHPLLFKSTARRARGSILGIWAVSLAIMVPQAAVME
CSSVLPELANRTRLFSVCDERWADDLYPKIYHSCFFIVTYLAPLGL
MAMAYFQIFRKLWGRQIPGTTSALVRNWKRPSDQLGDLEQGLSGEP
QPRGRAFLAEVKQMRARRKTAKMLMVVLLVFALCYLPISVLNVLKR
VEGMFRQASDREAVYACETFSHWLVYANSAANPIIYNFLSGKFREQ
FKAAFSCCLPGLGPCGSLKAPSPRSSASHKSLSLQSRCSISKISEH
VVLTSVTTVLP

As used herein the term “orexin-B” has its general meaning in the art and refers to the amino acid sequence as shown by SEQ ID NO:2.

Orexin-B_homo sapiens (SEQ ID NO: 2):
RSGPPGLQGRLQRLLQASGNHAAGILTM

According to the invention the polypeptides of the present invention are OX1R agonist. As used herein, the term “OX1R agonist” refers to any compound natural or not that is able to bind to OX1R and promotes OX1R activity which consists of activation of signal transduction pathways involving recruitment of SHP-2 and the induction of apoptosis of the cell, independently of transient calcium release. Agonistic activity of the polypeptide is assessed by any assay well known in the art. Typically, the apoptosis assay typically involve use of CHO—S cells expressing recombinant native or mutated OX1R that are seeded and grown as described in the EXAMPLE. After 24 hr culture, cells are treated with or without the polypeptide to be tested. After 48 hr of treatment, adherent cells were harvested by TryplE (Life Technologies, Saint Aubin, France). Apoptosis is then determined using the Guava PCA system and the Guava nexin kit as previously described (Voisin et al., 2008). Results are expressed as the percentage of apoptotic annexin V-phycoerythrin (PE)-positive cells. According to the invention, the polypeptide of the present invention keeps the same activity than Orexin-B. Typically, the apoptosis induction (EC50) of the polypeptide of the present invention ranges from 10 nM to 110 nM. More particularly, the apoptosis induction (EC50) of the polypeptide of the present invention ranges from 10 nM to 50 nM. More particularly, the apoptosis induction (EC50) of the polypeptide of the present invention ranges from 15 nM to 30 nM.

In some embodiments, the polypeptide of the present invention comprises the amino acid sequence ranging from the amino acid residue at position 6 to the amino acid residue at position 28 in SEQ ID NO:2 wherein at least one amino acid residue position 6, 7, 8, 9, 10, 12, 13, 14, 19, 21 or 23 is substituted and the amino acid residues at position 11; 15; 16; 17; 18; 20; 22; 24; 25; 26; 27; and 28 are not deleted or substituted.

As used herein, the term “substitution” means that a specific amino acid residue at a specific position is removed and another amino acid residue is inserted into the same position.

In some embodiments, the amino acid residue at position 6, 7, 8, 9, 10, 12, 13, 14, 19, 21, or 23 is substituted by an alanine.

In some embodiments, the substitution is a conservative substitution. In the context of the present invention, a “conservative substitution” is defined by substitutions within the classes of amino acids reflected as follows:

Aliphatic residues I, L, V, and M

Cycloalkenyl-associated residues F, H, W, and Y

Hydrophobic residues A, C, F, G, H, I, L, M, R, T, V, W, and Y

Negatively charged residues D and E

Polar residues C, D, E, H, K, N, Q, R, S, and T

Positively charged residues H, K, and R

Small residues A, C, D, G, N, P, S, T, and V

Very small residues A, G, and S

Residues involved in turn A, C, D, E, G, H, K, N, Q, R, S, P, and formation T

Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in the polypeptide of the present invention as compared to the native sequence of Orxin-B. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1).

In some embodiments, the polypeptide of the present invention comprises 1, 2, 3, 4, 5, 6, 7, 8, 9, 10 or 11 substitutions in the amino acid sequence ranging from the amino acid residue at position 6 to the amino acid residue at position 28 in SEQ ID NO:2.

In some embodiments, the methionine residue at position 28 is amidated. As used herein, the term “amidation” has its general meaning in the art and refers to the process consisting of producing an amide moiety.

In some embodiments, the polypeptide of the present invention is extended by at least one amino acid. In some embodiments, the polypeptide of the present invention is extended by at least one glycine. In said embodiments, the methionine at position 28 is not necessarily amidated.

In some embodiments, the polypeptide of the present invention is fused to a heterologous polypeptide to form a fusion protein. As used herein, a “fusion protein” comprises all or part (typically biologically active) of a polypeptide of the present invention operably linked to a heterologous polypeptide (i.e., a polypeptide other than the same polypeptide). Within the fusion protein, the term “operably linked” is intended to indicate that the polypeptide of the present invention and the heterologous polypeptide are fused in-frame to each other. The heterologous polypeptide can be fused to the N-terminus or C-terminus of the polypeptide of the present invention. In some embodiment, the heterologous polypeptide is fused to the C-terminal end of the polypeptide of the present invention.

In some embodiments, the polypeptide of the present invention and the heterologous polypeptide are fused to each other directly (i.e. without use of a linker) or via a linker. The linker is typically a linker peptide and will, according to the invention, be selected so as to allow binding of the polypeptide to the heterologous polypeptide. Suitable linkers will be clear to the skilled person based on the disclosure herein, optionally after some limited degree of routine experimentation. Suitable linkers are described herein and may—for example and without limitation—comprise an amino acid sequence, which amino acid sequence preferably has a length of 2 or more amino acids. Typically, the linker has 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. However, the upper limit is not critical but is chosen for reasons of convenience regarding e.g. biopharmaceutical production of such fusion proteins. The linker sequence may be a naturally occurring sequence or a non-naturally occurring sequence. If used for therapeutical purposes, the linker is preferably non-immunogenic in the subject to which the fusion protein of the present invention is administered. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)3.

In some embodiments, the polypeptide of the present invention is fused to an immunoglobulin domain. For example the fusion protein of the present invention may comprise a polypeptide of the present invention that is fused to an Fc portion (such as a human Fe) to form an immunoadhesin. As used herein, the term “immunoadhesin” designates antibody-like molecules which combine the binding specificity of a heterologous protein (an “adhesin” which is able to bind to OX1R) with the effector functions of immunoglobulin constant domains. Structurally, the immunoadhesins comprise a fusion of the polypeptide of the present invention and an immunoglobulin constant domain sequence. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM. The immunoglobulin sequence typically, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fe) sequence, the binding specificity of interest can be achieved using entirely human components. Such immunoadhesins are minimally immunogenic to the patient, and are safe for chronic or repeated use. In some embodiments, the Fc region is a native sequence Fc region. In some embodiments, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3.

The polypeptides of the present invention can exhibit post-translational modifications, including, but not limited to glycosylations, (e.g., N-linked or O-linked glycosylations), myristylations, palmitylations, acetylations and phosphorylations (e.g., serine/threonine or tyrosine). In some embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

The polypeptides of the present invention may be produced by any suitable means, as will be apparent to those of skill in the art. In order to produce sufficient amounts of polypeptides or functional equivalents thereof for use in accordance with the present invention, expression may conveniently be achieved by culturing under appropriate conditions recombinant host cells containing the polypeptide of the present invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells, HeLa cells, baby hamster kidney cells and many others. Bacteria are also preferred hosts for the production of recombinant protein, due to the ease with which bacteria may be manipulated and grown. A common, preferred bacterial host is E coli. Methods for producing amidated polypeptide are well known in the art and typically involve use of amidation enzyme. As used herein, the term “amidation enzyme” is defined as the enzymes which can convert the carboxyl group of a polypeptide to an amide group. Enzymes capable of C-terminal amidation of peptides have been known for a long time (Eipper et al. Mol. Endocrinol. 1987 November; 1 (11): 777). Examples of amidating enzymes include peptidylglycine α-monooxygenase (EC 1.14.17.3), herein referred to as PAM, and peptidylamidoglycolate lyase (EC 4.3.2.5), herein referred to as PGL. The preparation and purification of such PAM enzymes is familiar to the skilled worker and has been described in detail (M. Nogudi et al. Prot. Expr. Purif. 2003, 28: 293). An alternative to the “in vitro” amidation by means of PAM emerges when the enzyme is coexpressed in the same host cell with the precursor protein to be amidated (i.e the fusion protein of the present invention). This is achieved by introducing a gene sequence which codes for a PAM activity into the host cell under the control of a host-specific regulatory sequence. This expression sequence can either be incorporated stably into the respective chromosomal DNA sequence, or be present on a second plasmid parallel to the expression plasmid for the target protein (i.e. fusion protein of the present invention), or be integrated as second expression cassette on the same vector, or be cloned in a polycistronic expression approach in phase with the gene sequence which encodes the target protein (i.e. fusion protein of the present invention) under the control of the same promoter sequence. A further method for amidation is based on the use of protein-specific self-cleavage mechanisms (Cottingham et al. Nature Biotech. Vol. 19, 974-977, 2001). The amidation processes described above start from a C terminus of the target peptide which is extended by at least one amino acid glycine or alternatively interim peptide. Alternative methods, are also described in WO2007036299.

Accordingly, in some embodiments, the nucleic acid sequence encoding for the orexin polypeptide is chosen to allow the amidation of said orexin polypeptide and thus may comprise additional codons that will code for a glycine-extended precursor. Typically, the glycine-extended precursor resembles YGXX, where Y represents the amino acid that shall be amidated and X represents any amino acid so that the amidation enzyme (e.g. PAM) catalyzes the production of the amidated polypeptide from said glycine-extended precursor. In some embodiments, the glycine-extended precursor is MG, MGR, MGRR, MGK or MGKK.

The polypeptide of the present invention (fused or not to the heterologous polypeptide) is produced by any technique known in the art, such as, without limitation, any chemical, biological, genetic or enzymatic technique, either alone or in combination. For example, knowing the amino acid sequence of the desired sequence, one skilled in the art can readily produce said polypeptide (fused or not to the heterologous polypeptide), by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, the polypeptide of the present invention (fused or not to the heterologous polypeptide) can be synthesized by recombinant DNA techniques well-known in the art. For example, the polypeptide of the present invention (fused or not to the heterologous polypeptide) can be obtained as DNA expression products after incorporation of DNA sequences encoding the polypeptide (fused or not to the heterologous polypeptide) into expression vectors and introduction of such vectors into suitable eukaryotic or prokaryotic hosts that will express the desired polypeptide, from which they can be later isolated using well-known techniques. A variety of expression vector/host systems may be utilized to contain and express the polypeptide of the present invention (fused or not to the heterologous polypeptide). These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein. In the recombinant production of the polypeptide of the present invention (fused or not to the heterologous polypeptide), it would be necessary to employ vectors comprising polynucleotide molecules for encoding said polypeptide. Methods of preparing such vectors as well as producing host cells transformed with such vectors are well known to those skilled in the art. The polynucleotide molecules used in such an endeavour may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation. The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed. The choice of a suitable expression vector for expression of polypeptide of the present invention will of course depend upon the specific host cell to be used, and is within the skill of the ordinary artisan. Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. Typically, the nucleotide sequences are operably linked when the regulatory sequence functionally relates to the DNA encoding the protein of interest (e.g., a polypeptide). Thus, a promoter nucleotide sequence is operably linked to a given DNA sequence if the promoter nucleotide sequence directs the transcription of the sequence. They may then, if necessary, be purified by conventional procedures, known in themselves to those skilled in the art, for example by fractional precipitation, in particular ammonium sulphate precipitation, electrophoresis, gel filtration, affinity chromatography, etc. In particular, conventional methods for preparing and purifying recombinant proteins may be used for producing the proteins in accordance with the invention.

A further object of the present invention relates to a nucleic acid molecule which encodes for a polypeptide of the present invention (fused or not to the heterologous polypeptide).

As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the nucleic acid molecule of the present invention is included in a suitable vector, such as a plasmid, cosmid, episome, artificial chromosome, phage or a viral vector. So, a further object of the invention relates to a vector comprising a nucleic acid encoding for a polypeptide of the invention (fused or not to the heterologous polypeptide). Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

A further object of the present invention relates to a host cell transformed with the nucleic acid molecule of the present invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. For instance, as disclosed above, for expressing and producing the polypeptide of the present invention, prokaryotic cells and, in particular E. coli cells, will be chosen. Actually, according to the invention, it is not mandatory to produce the polypeptides of the present invention in a eukaryotic context that will favour post-translational modifications (e.g. glycosylation). Typically, the host cell may be suitable for producing the polypeptide of the present invention (fused or not to the heterologous polypeptide) as described above. In some embodiments, the host cells is isolated from a mammalian subject who is selected from a group consisting of: a human, a horse, a dog, a cat, a mouse, a rat, a cow and a sheep. In some embodiments, the host cell is a human cell. In some embodiments, the host cell is a cell in culture. The cells may be obtained directly from a mammal (preferably human), or from a commercial source, or from tissue, or in the form for instance of cultured cells, prepared on site or purchased from a commercial cell source and the like. In some embodiments, the host cell is a mammalian cell line (e.g., Vero cells, CHO cells, 3T3 cells, COS cells, etc.).

In another aspect, the present invention relates to the polypeptide of the present invention, as defined in any aspect or embodiment herein, for use as a medicament.

In another aspect, the present invention relates to a method of treating cancer in a subject in need thereof comprising administering the subject with a therapeutically effective amount of a polypeptide of the present invention.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present invention contemplate any one or more of these aspects of treatment.

Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyo sarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

In some embodiments, the subject suffers from an epithelial cancer. As used herein, the term “epithelial cancer” refers to any malignant process that has an epithelial origin. Examples of epithelial cancers include, but are not limited to, a gynecological cancer such as endometrial cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer colorectal cancer and liver cancer. An epithelial cancer may be at different stages as well as varying degrees of grading. In some embodiments, the epithelial cancer is selected from the group consisting of breast cancer, prostate cancer, lung cancer, pancreatic cancer, bladder cancer colorectal cancer and ovarian cancer. In some embodiments, the epithelial cancer is a colorectal cancer. In some embodiments, the epithelial cancer is a liver cancer, in particular a hepatocellular carcinoma. In some embodiments, the epithelial cancer is breast cancer. In some embodiments, the epithelial cancer is ovarian cancer. In some embodiments, the epithelial cancer is prostate cancer, in particular advanced prostate cancer. In some embodiments, the epithelial cancer is lung cancer. In some embodiments, the epithelial cancer is head and neck cancer. In some embodiments, the epithelial cancer is head and neck squamous cell carcinoma.

As used herein the term “pancreatic cancer” or “pancreas cancer” as used herein relates to cancer which is derived from pancreatic cells. In particular, pancreatic cancer included pancreatic adenocarcinoma (e.g., pancreatic ductal adenocarcinoma) as well as other tumors of the exocrine pancreas (e.g., serous cystadenomas), acinar cell cancers, intraductal papillary mucinous neoplasms (IPMN) and pancreatic neuroendocrine tumors (such as insulinomas).

As used herein the term “hepatocellular carcinoma” has its general meaning in the art and refers to the cancer developed in hepatocytes. In general, liver cancer indicates hepatocellular carcinoma in large. HCC may be caused by an infectious agent such as hepatitis B virus (HBV, hereinafter may be referred to as HBV) or hepatitis C virus (HCV, hereinafter may be referred to as HCV). In some embodiments, HCC results from alcoholic steatohepatitis or non-alcoholic steatohepatitis (hereinafter may be abbreviated to as “NASH”). In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or Ml tumor (distant metastasis). In some embodiments, the HCC is, according to AJCC (American Joint Commission on Cancer) staging criteria, stage T1, T2, T3, or T4 HCC.

As used herein the term “advanced prostate cancer” has its general meaning in the art. “Castration resistant prostate cancer,” “CaP,” “androgen-receptor dependent prostate cancer,” “androgen-independent prostate cancer,” are used interchangeably to refer to prostate cancer in which prostate cancer cells “grow” {i.e., increase in number) in the absence of androgens and/or in the absence of expression of androgen receptors on the cancer cells.

As used herein, the term “therapeutically effective amount” refers to an amount effective, at dosages and for periods of time necessary, to achieve a desired therapeutic result. A therapeutically effective amount of a polypeptide of the present invention may vary according to factors such as the disease state, age, sex, and weight of the individual, and the ability of the polypeptide of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the polypeptide of the present invention depend on the disease or condition to be treated and may be determined by the persons skilled in the art. A physician having ordinary skill in the art may readily determine and prescribe the effective amount of the pharmaceutical composition required. For example, the physician could start doses of the polypeptide of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. The ability of a compound to inhibit cancer may, for example, be evaluated in an animal model system predictive of efficacy in human tumors. Alternatively, this property of a composition may be evaluated by examining the ability of the compound to inhibit cell growth or to induce cytotoxicity by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of a polypeptide of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a polypeptide of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by measuring the level of OX1R in a sample containing tumor cells, by visualization of the disease area, or by other diagnostic methods described further herein, e.g. by performing one or more PET-CT scans, for example using a labeled polypeptide of the present invention, fragment or mini-antibody derived from the polypeptide of the present invention. If desired, an effective daily dose of a pharmaceutical composition may be administered as two, three, four, five, six or more sub-doses administered separately at appropriate intervals throughout the day, optionally, in unit dosage forms. In some embodiments, the polypeptides of the present invention are administered by slow continuous infusion over a long period, such as more than 24 hours, in order to minimize any unwanted side effects. An effective dose of a polypeptide of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a compound of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

The present invention also provides for therapeutic applications where a polypeptide of the present invention is used in combination with at least one further therapeutic agent for treating cancer. Such administration may be simultaneous, separate or sequential. For simultaneous administration the agents may be administered as one composition or as separate compositions, as appropriate.

The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

In one aspect, the further therapeutic agent is at least one antibody which binds another target such as, e.g., CC1, CD5, CD8, CD14, CD15, CD19, CD21, CD22, CD23, CD25, CD30, CD33, CD37, CD38, CC10, CC10L, CC16, CD52, CD54, CD80, CD126, B7, MUC1, tenascin, HM 1.24, or HLA-DR. For example, the second antibody may bind to a B cell antigen, including, but not limited to CD20, CD19, CD21, CD23, CD38, CC16, CD80, CD138, HLA-DR, CD22, or to another epitope on OX1R. In some embodiments, the second antibody binds vascular endothelial growth factor A (VEGF-A). In some embodiments, the polypeptide of the present invention is for use in combination with a specific therapeutic antibody. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference].

In some embodiments, the polypeptide of the present invention is used in combination with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylamine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the polypeptide of the present invention is used in combination with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the polypeptide of the present invention is used in combination with a HER inhibitor. In some embodiments, the HER inhibitor is an EGFR inhibitor. GFR inhibitors are well known in the art (Inhibitors of erbB-1 kinase; Expert Opinion on Therapeutic Patents December 2002, Vol. 12, No. 12, Pages 1903-1907, Susan E Kane. Cancer therapies targeted to the epidermal growth factor receptor and its family members. Expert Opinion on Therapeutic Patents February 2006, Vol. 16, No. 2, Pages 147-164. Peter TrOX1Rer Tyrosine kinase inhibitors in cancer treatment (Part II). Expert Opinion on Therapeutic Patents December 1998, Vol. 8, No. 12, Pages 1599-1625). Examples of such agents include antibodies and small organic molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small organic molecules that bind to EGFR include ZD1839 or Gefitinib (IRESSA™; Astra Zeneca); CP-358774 or erlotinib (TARCEVA™; Genentech/OSI); and AG1478, AG1571 (SU 5271; Sugen); EMD-7200. In some embodiments, the HER inhibitor is a small organic molecule pan-HER inhibitor such as dacomitinib (PF-00299804). In some embodiments, the HER inhibitor is selected from the group consisting of cetuximab, panitumumab, zalutumumab, nimotuzumab, erlotinib, gefitinib, lapatinib, neratinib, canertinib, vandetanib, afatinib, TAK-285 (dual HER2 and EGFR inhibitor), ARRY334543 (dual HER2 and EGFR inhibitor), Dacomitinib (pan-ErbB inhibitor), OSI-420 (Desmethyl Erlotinib) (EGFR inhibitor), AZD8931 (EGFR, HER2 and HER3 inhibitor), AEE788 (NVP-AEE788) (EGFR, HER2 and VEGFR 1/2 inhibitor), Pelitinib (EKB-569) (pan-ErbB inhibitor), CUDC-101 (EGFR, HER2 and HDAC inhibitor), XL647 (dual HER2 and EGFR inhibitor), BMS-599626 (AC480) (dual HER2 and EGFR inhibitor), PKC412 (EGFR, PKC, cyclic AMP-dependent protein kinase and S6 kinase inhibitor), BIBX1382 (EGFR inhibitor) and AP261 13 (ALK and EGFR inhibitor). The inhibitors cetuximab, panitumumab, zalutumumab, nimotuzumab are monoclonal antibodies. erlotinib, gefitinib, lapatinib, neratinib, canertinib, vandetanib and afatinib are tyrosine kinase inhibitors.

In some embodiments, the polypeptide of the present invention is used in combination with a c-Met inhibitor. In some embodiments the c-Met inhibitor is an anti-c-Met antibody. In some embodiments, the anti-c-met antibody is MetMAb (onartuzumab) or a biosimilar version thereof. MetMAb is disclosed in, for example, WO2006/015371; Jin et al, Cancer Res (2008) 68:4360. Other anti-c-met antibodies suitable for use in the methods of the present invention are described herein and known in the art. For example, anti-c-met antibodies disclosed in WO05/016382 (including but not limited to antibodies 13.3.2, 9.1.2, 8.70.2, 8.90.3); an anti-c-met antibodies produced by the hybridoma cell line deposited with ICLC number PD 03001 at the CBA in Genoa, or that recognizes an epitope on the extracellular domain of the β chain of the HGF receptor, and said epitope is the same as that recognized by the monoclonal antibody); anti-c-met antibodies disclosed in WO2007/126799 (including but not limited to 04536, 05087, 05088, 05091, 05092, 04687, 05097, 05098, 05100, 05101, 04541, 05093, 05094, 04537, 05102, 05105, 04696, 04682); anti c-met antibodies disclosed in WO2009/007427 (including but not limited to an antibody deposited at CNCM, Institut Pasteur, Paris, France, on Mar. 14, 2007 under the number 1-3731, on Mar. 14, 2007 under the number 1-3732, on Jul. 6, 2007 under the number 1-3786, on Mar. 14, 2007 under the number 1-3724; an anti-c-met antibody disclosed in 20110129481; an anti-c-met antibody disclosed in US20110104176; an anti-c-met antibody disclosed in WO2009/134776; an anti-c-met antibody disclosed in WO2010/059654; an anti-c-met antibody disclosed in WO2011020925 (including but not limited to an antibody secreted from a hybridoma deposited at the CNCM, Institut Pasteur, Paris, France, on Mar. 12, 2008 under the number 1-3949 and the hybridoma deposited on Jan. 14, 2010 under the number 1-4273). In some embodiments, the cMET inhibitor is selected from the group consisting of K-252a; SU-11274; PHA-665752 and other cMET inhibitors described in WO 2002/096361; AM7; AMG-208 and other cMet inhibitors described in WO 2009/091374; JNJ-38877605 and other cMet inhibitors described in WO 2007/075567; MK-2461 and other cMet inhibitors described in WO 2007/002254 and/or WO 2007/002258; PF-04217903 and other cMet inhibitors described in WO 2007/132308; BMS 777607; GSK 136089 (also known as XL-880 and Foretinib) and other cMET inhibitors described in WO 2005/030140; BMS 907351 (also known as XL-184); EMD 1214063; LY 2801653; ARQ 197; MK 8033; PF 2341066 and other cMET inhibitors described in WO 2006/021881; MGCD 265; E 7050; MP 470; SGX 523; cMet inhibitors described in Kirin J. J. Cui, Inhibitors targeting hepatocyte growth factor receptor and their potential therapeutic applications. Expert Opin. Ther. Patents 2007; 17: 1035-45; cMet inhibitors described in WO 2008/103277; cMet inhibitors described in WO 2008/008310; cMet inhibitors described in WO 2007/138472; cMet inhibitors described in WO 2008/008539; cMet inhibitors described in WO 2009/007390; cMet inhibitors described in WO 2009/053737; cMet inhibitors described in WO 2009/024825; cMet inhibitors described in WO 2008/071451; cMet inhibitors described in WO 2007/130468; cMet inhibitors described in WO 2008/051547; cMet inhibitors described in WO 2008/053157; cMet inhibitors described in WO 2008/017361; WO 2008/145242; WO2008/145243; WO 2008/148449; WO 2009/007074; WO 2009/006959; WO 2009/024221; WO 2009/030333; and/or WO 2009/083076; cMet inhibitors described in WO 2009/093049; cMet inhibitors described in US 2008/039457; cMet inhibitors described in WO 2007/149427; cMet inhibitors described in WO 2007/050309; cMet inhibitors described in WO 2009/056692; cMet inhibitors described in WO 2009/087305; cMet inhibitors described in US 2009/197864; cMet inhibitors described in US 2009/197862; cMet inhibitors described in US 2009/156594; cMet inhibitors described in WO 2008/124849; cMet inhibitors described in WO 2008/067119; cMet inhibitors described in WO 2007/064797; cMet inhibitors described in WO 2009/045992; cMet inhibitors described in WO 2008/088881; cMet inhibitors described in WO 2007/081978; cMet inhibitors described in WO 2008/079294; cMet inhibitors described in WO 2008/079291; cMet inhibitors described in WO 2008/086014; cMet inhibitors described in WO 2009/033084; cMet inhibitors described in WO 2007/059202; cMet inhibitors described in US 2009/170896; cMet inhibitors described in WO 2009/077874 and/or WO 2007/023768; cMet inhibitors described in WO 2008/049855; cMet inhibitors described in WO 2009/026717; and cMet inhibitors described in WO 2008/046216.

In some embodiments, the polypeptide of the present invention is used in combination with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

Combination compositions and combination administration methods of the present invention may also involve “whole cell” and “adoptive” immunotherapy methods. For instance, such methods may comprise infusion or re-infusion of immune system cells (for instance tumor-infiltrating lymphocytes (TILs), such as CC1+ and/or CD8+ T cells (for instance T cells expanded with tumor-specific antigens and/or genetic enhancements), antibody-expressing B cells or other antibody-producing or -presenting cells, dendritic cells (e.g., dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887, July 2001), optionally admixed with adjuvants such as alum, may also be components in such methods and combination compositions.

In some embodiments, the polypeptide of the present invention is used in combination with radiotherapy. Radiotherapy may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

For administration, the polypeptide of the present invention is formulated as a pharmaceutical composition. A pharmaceutical composition comprising a polypeptide of the present invention can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the present invention can be formulated for a topical, oral, parenteral, intranasal, intravenous, intramuscular, subcutaneous or intraocular administration and the like.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the polypeptide of the present invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A polypeptide of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The polypeptides of the present invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g. tablets or other solids for oral administration; time release capsules; and any other form currently used.

In some embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospho lipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention will be further illustrated by the following figures and examples. However, these examples and figures should not be interpreted in any way as limiting the scope of the present invention.

### FIGURES

FIG. 1: Binding characteristics (top panel) and pro-apoptotic properties (bottom panel) of wt human OxB and peptide analogs. Single amino acids in the wt OxB sequence were replaced by L-alanine or L-leucine as mentioned at the x-axis. Top panel: wt OX1R were stably expressed in CHO—S cells, and competitive inhibition of ¹²⁵I-OxA binding by unlabeled mutants was analyzed. The concentration of each mutant that half-maximally inhibited specific ¹²⁵I-OxA binding (IC₅₀) was determined and the ratio IC₅₀mutant/IC₅₀wt was calculated; Bottom panel: Pro-apoptotic activity of wt OxB and peptide analogs determined in CHO—S—OX1R cells. The concentration of each mutant that half-maximally induced cellular apoptosis (EC₅₀) was determined and the ratio EC₅₀mutant/EC₅₀wt was calculated.

FIG. 2: Correlation between the EC₅₀ values and IC₅₀ values determined for native OxB and all singly-substituted mutants of OxB. A: correlation between the EC₅₀ values and Ki values determined for the IP3 and binding assays in CHO—S cells stably expressing recombinant OX1R (CHO—S-OX1R); B: correlation between the EC₅₀ values and IC₅₀ values determined for the apoptosis and binding assays in CHO—S-OX1R. Note the S18A, N20A and T27A mutants for which the EC₅₀ for inducing cellular apoptosis was much higher than their IC₅₀ for binding. See Table I for details.

FIG. 3: Specific ¹²⁵I-OxA binding to CHO—S-OX1R cells in the presence of increasing concentrations of unlabeled OxB, S18A/N20A and S18A/T27A peptides (A) and determination of the inhibition of cellular growth induced by increasing concentrations of OxB, S18A/N20A (B,C) and S18A/T27A (B,D) peptides in CHO—S-OX1R cells. A: CHO—S-OX1R cells were incubated with the indicated concentrations of OxB (), S18A/N20A (▴) and S18A/T27A (Δ). Results are expressed as the percentage of radioactivity specifically bound in the absence of added unlabeled peptide; B: CHO—S-OX1R cells were incubated with the indicated concentrations of OxB (), S18A/N20A (▴) and S18A/T27A (Δ), and cells were counted after 48 hr incubation. Results are expressed as the percentage of total viable cells; C and D: the indicated concentrations of OxB were incubated together with the following concentrations of S18A/N20A mutant (C): 0 (), 10 nM (▴) 100 nM (Δ), 1 μM (▾), 10 μM (▪) or S18A/T27A mutant (D): 0 (◯), 10 nM (⋄), 100 nM (Δ), 1 μM (∇), 10 μM (□). Each point is the mean of three separate experiments. For the sake of clarity, standard errors are not indicated. They were always below 15% of mean values.

FIG. 4: Specific ¹²⁵I-OxA binding to HEK-OX1R cells in the presence of increasing concentrations of unlabeled OxB OxBGly29 peptides. HEK-OX1R cells were incubated with the indicated concentrations of OxB () and OxBGly29 (▪). Results are expressed as the percentage of radioactivity specifically bound in the absence of added unlabeled peptide.

FIG. 5: Determination of the inhibition of cellular growth induced by increasing concentrations of OxB and OxBGly29 peptides in HEK-OX1R cells. HEK-OX1R cells were incubated with the indicated concentrations of OxB () and OxBGly29 (▪), and living cells were counted after 48 hr incubation. Results are expressed as the percentage of total viable cells.

### EXAMPLES

### Example 1

Material & Methods

Materials—

Enzymes, vectors and culture medium were obtained from Life Technologies (Saint Aubin, France). All OxB analogs were obtained by custom solid-phase synthesis from GL Biochem (Shanghai, China). ¹²⁵I-OxA was prepared and purified as previously described (Voisin et al., 2008). All other highly purified chemicals were from Sigma-Aldrich (Saint Quentin-Fallavier, France).

Stable Expression of cDNA Encoding Human OX1R, OX2R and Mutated OX1R in CHO—S Cells—

Full-length OX1R cDNAs were cloned into the expression vector pEYFP-N2 in fusion at the C-terminus with the yellow fluorescent protein (YFP) as described (El Firar et al., 2009). The plasmid encoding human OX1R was stably transfected into CHO—S cells (ECACC 85050302) using X-tremeGENE (ROCHE Diagnostics, Meylan, France) according to the manufacturer's protocol. Transfected CHO—S cells were selected in the presence of 1 mg/ml geneticin (G418, Life Technologies, Saint Aubin, France) for 2 weeks, and cloned (Ceraudo et al., 2012). OX1R mutants shown in Table II were obtained by site-directed mutagenesis as previously described (Ceraudo et al., 2012). Each mutation was verified by sequencing. The recombinant mutants were stably expressed in CHO—S cells as described above.

Cell Culture and Radioreceptor Assays—

CHO—S cells expressing recombinant native or mutated OX1R were grown as previously described (El Firar et al., 2009) and maintained at 37° C. in a humidified 5% CO₂/air incubator. ¹²⁵I-orexia-A (74 TBq/mmol) was prepared as previously described (El Firar et al., 2009). Binding of ¹²⁵I-OxA to subconfluent cells was conducted as described (El Firar et al., 2009). It should be noted that OxA is used for this assay, as it contains a tyrosine residue that can be iodinated, which is not the case of OxB. Briefly, cells were incubated for 30 min at room temperature in 300 μl of 20 mM Tris/EDTA/saline (TES) binding buffer (pH 7.4) containing 0.5% (w/v) BSA, 5 mM KCL, 1 mM CaCl₂, 1.2 mM MgCl₂, 0.44 mM KH₂PO₄, 4.2 mM NaHCO₃, 10 mM glucose, 1 mM probenecid and 0.001% (v/v) Tween 20, in the presence of 0.25 nM ¹²⁵I-OxA with or without peptide analogs to be tested. Free peptides were removed by 2 cell washing cycles with PBS. The nonspecific binding was determined in the presence of 1 μM unlabeled OxB, and represented <5% of the total binding. Data were analyzed using the GraphPad software (http://www.graphpad.com).

Apoptosis and Inositol Phosphate (InsP) Assays—

CHO—S cells expressing recombinant native or mutated OX1R were seeded and grown as described above. After 24 hr culture, cells were treated with or without peptide analogs to be tested at the concentration indicated in the figure legends. After 48 hr of treatment, adherent cells were harvested by TriplE (Life Technologies, Saint Aubin, France). Apoptosis was determined using the Guava PCA system and the Guava nexin kit as previously described (Voisin et al., 2008). Results are expressed as the percentage of apoptotic annexin V-phycoerythrin (PE)-positive cells.

Subconfluent cells were labeled with [myo-3H] inositol (3.15 TBq/mmol) (TRK883; GE Heathcare, Les Ullis, France) for 24 hr in standard culture medium. Labeled cells were then incubated for 30 min at 37° C. in TES binding buffer (pH 7.4) containing 20 mM lithium chloride with or without increasing concentration of the peptide analogs to be tested. Cells were then treated with ice-cold formic acid, and total InsP was separated from free [myo-3H] inositol using column chromatography as previously reported (Rouet-Benzineb et al., 2004).

3D-Modeling of Human OX1R and Docking of C-Terminal End of OxB in the 3D-Model of OX1R—

The OX1R sequence was aligned with the sequence of the human β2 adrenergic receptor active form (pdb: 3SN6), another GPCR for which the 3D structure has already been resolved (Rasmussen et al., 2011). Sequence alignment was performed with ClustalW, and manually refined (Ceraudo et al., 2012). Based on sequence alignment, 100 homology models were built with the Modeller 9v11 software (http://www.salilab.org/modeller/). An objective function for each model was calculated to select the best score (Ceraudo et al., 2012). Models were refined by energy minimization (AMBER forcefield). Docking simulations were performed using the NMR structure of the OxB C-terminus (pdb: 1CQ0). Briefly, the binding pocket was determined using Fpocket webserver (Le Guilloux et al., 2009). The largest pocket, localized at the top of the transmembrane domains of OX1R, was selected. The docking of the OxB C-terminus (sequence 20-28) into OX1R was performed with the HADDOCK web-online platform (http://haddock.chem.uu.nl/) (Dominguez et al., 2003) under distance restraints (<6 Å) between the residues of the OxB C-terminus and the putative interacting residues that delineate the binding pocket as determined above. Intermolecular distances were defined with a maximum value of 6 Å between any atoms of each interacting residues. The best structure was selected using the HADDOCK score and buried surface area. Energy minimization of the best structure was performed with CHARMM29 (Ceraudo et al., 2008). Molecular dynamics simulations using CHARMM29 force field were applied in VEGAZZ (www.ddl.unimi.it/vegazz/) and NAMD software for 1 ns to check general reasonability of the formed complex structure (Phillips et al., 2005). Three-dimensional representations of the interaction between the C-terminal region of OxB and the 3D-model of OX1R were drawn using PyMOL v1.6 software (http://www.pymol.org).

Results

Structure-Function Relationship of Orexin-B.

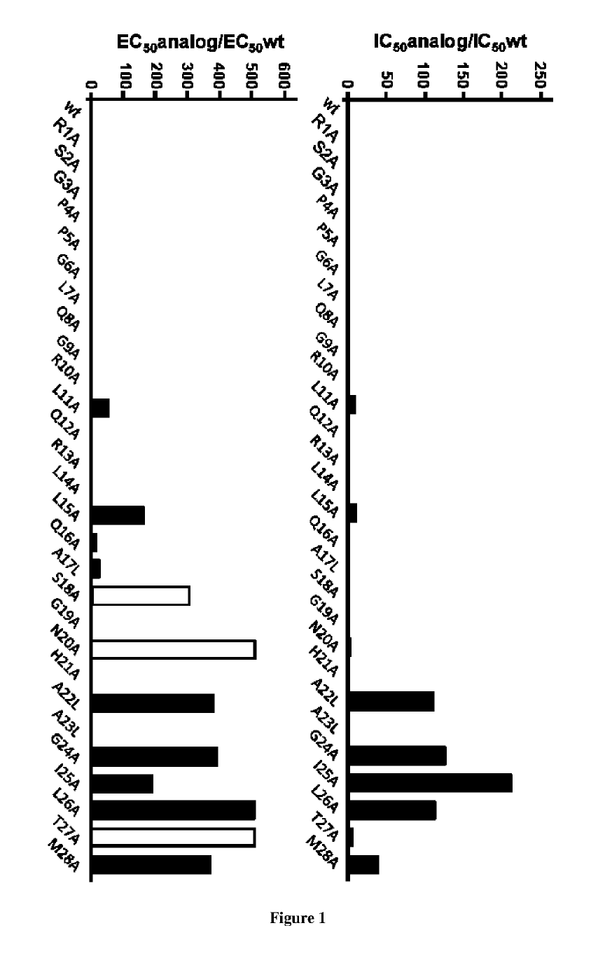

Previous reports have clearly demonstrated that OxA and OxB induce mitochondrial apoptosis in colonic cancer cell lines as HT-29, LoVo, and other cell lines, which express OX1R, but not OX2R (Voisin et al., 2011). In order to identify the pharmacophore of OxB responsible for this effect, we used the alanine scanning technique (Nicole et al., 2000). Twenty-five mutants of OxB were synthesized in which the 25 residue-side chains were individually replaced with alanine. As the residues in position 17, 22 or 23 of the native peptide are alanine, three additional mutants were synthesized, substituting the alanine for a leucine residue. All mutants were tested for their interaction with human recombinant OX1R, stably expressed in CHO—S cells, by competitive inhibition of ¹²⁵I-OxA binding. The slopes of the mutant dose-response curves for inhibiting ¹²⁵-OxA binding were identical for all mutants (not shown). All competitor curves analyzed with the GraphPad software fitted to a monophasic binding pattern, indicating the presence of only one binding site. When the IC₅₀ of all mutants was analyzed, the largest decrease in affinity for OX1R occurred when A²², G²⁴, I²⁵, L²⁶ and M²⁸ were substituted for alanine or leucine, resulting in a 40- to 200-fold decrease (Table I and FIG. 1). Alanine substitution of L¹¹ and L¹⁵ resulted in significant decrease in IC₅₀, but by only 10-fold (Table I and FIG. 1). It should be noted that alanine substitutions at position 20 and 27 induced a weak decrease of affinity, i.e., <5-fold. Substitution at other positions, including S¹⁸, N²⁰ and T²⁷, either did not change the affinity or resulted only in decreases of less than one log (Table I and FIG. 1).

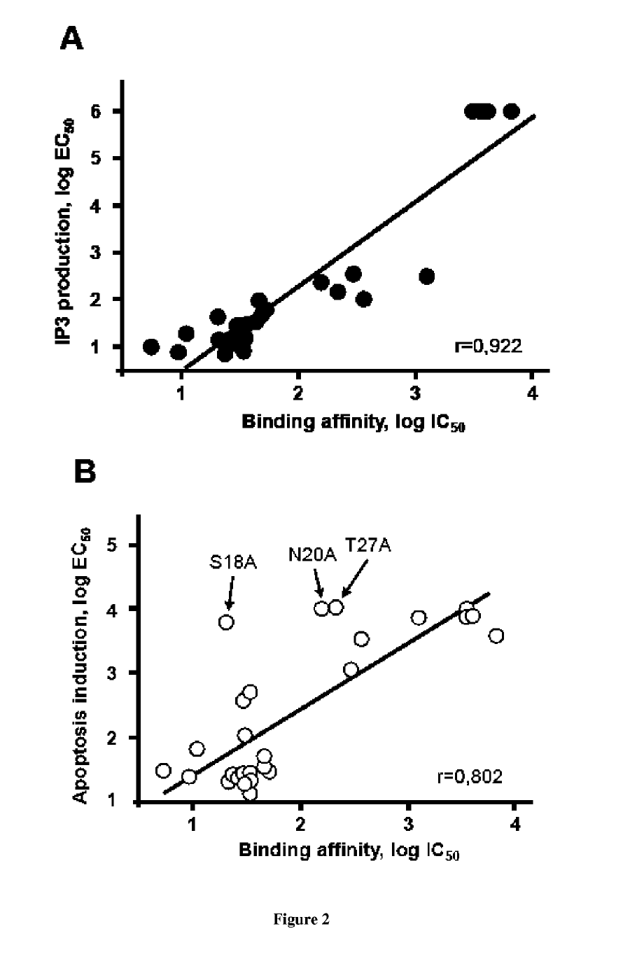

All OxB mutants were tested for their ability to stimulate IP3 production in CHO—S-OX1R cells (Table I). The dose-response curves of IP3 production for all mutants were identical to those of native OxB (not shown). There was a good correlation between the EC₅₀ for intracellular IP3 production and the IC₅₀ for the binding inhibition of ¹²⁵I-OxA to OX1R (Table I). A straight line (r=0.92) was obtained when plotting log EC₅₀ vs log IC₅₀, indicating that all mutants behaved as OX1R agonists, with identical or lower potencies than native OxB (FIG. 2). Taken together, these data demonstrate the importance of five crucial residues in the C-terminal sequence of OxB, i.e., A²², G²⁴, I²⁵, L²⁶ and M²⁸ (FIG. 1). Nevertheless, a minor contribution of the central part of the peptide is also to be noted, specifically the L¹¹ and L¹⁵ residues (Table I and FIG. 1). All mutants were then tested for their ability to trigger apoptosis in CHO—S-OX1R cells. As expected, alanine substitution of L¹¹, L¹⁵, A²², G²⁴, I²⁵, L²⁶ and M²⁸, which all play a role in OxB affinity and cellular IP3 production (see above), decreased peptide-induced apoptosis by 50- to 500-fold (Table I and FIG. 1).

Interestingly, a strong decrease in the ability to induce apoptosis occurred when S¹⁸, N²⁰ and T²⁷ were substituted for alanine (Table I and FIG. 1). Indeed, these substitutions produced at least a 100-fold drop in OxB-induced apoptosis (Table I and FIG. 1), even though they were able to bind to OX1R with relatively good affinity (Table I and FIG. 1). Similarly, although to a lesser extent, mutants Q16A and A17L, which bind to OX1R with a similar affinity as wt OxB, exhibited a decrease in pro-apoptotic activity by about 20-fold (Table I). Globally, a good correlation (r=0.80) between the EC₅₀ for induction of apoptosis and IC₅₀ for inhibiting ¹²⁵I-OxA binding was observed for all mutants, except for the S18A, N20A and T27A mutants (FIG. 2), suggesting that these peptides behaved as partial OX1R agonists. When they were omitted from the regression curve, the correlation coefficient improved (r=0.90), indicating a lack of correlation between OX1R affinity and apoptosis induction for the S18A, N20A and T27A mutants.

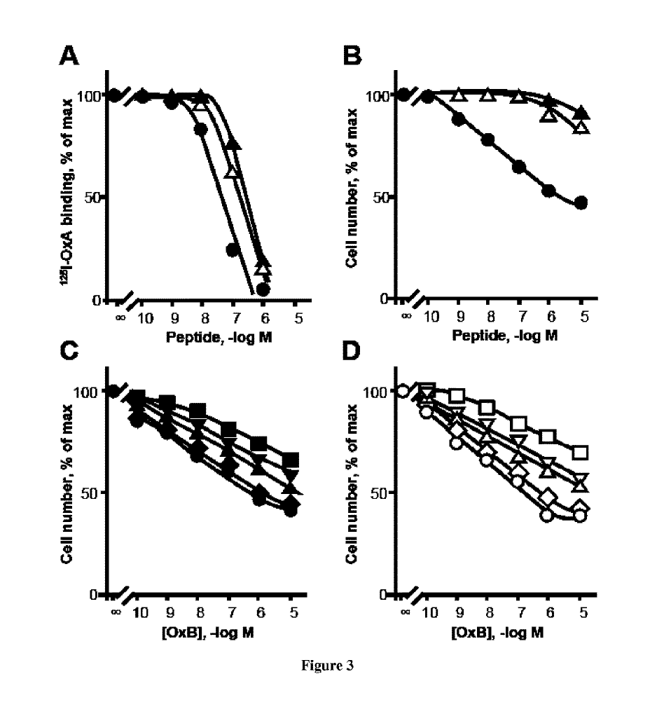

To confirm these observations, two new OxB mutants were synthetized with double substitution by alanine of the S¹⁸ and N²⁰ or S¹⁸ and T²⁷ residues. As shown in Table I and FIG. 3A, both double mutants slightly altered the ability of the peptide to interact with OX1R (IC₅₀=31.2 nM for wt vs 200.2 nM for S18A/N20A and 175 nM for S18A/T27A). In contrast, both strongly abolished the production of IP3 and the induction of apoptosis (Table I). Thus, these data suggest that the S18A/N20A and S18A/T27A mutants might be partial agonists. To confirm this, the ability of OxB to inhibit cell growth was measured in the presence or absence of either the S18A/N20A or S18A/T27A peptides. As shown in FIG. 3, OxB induced a strong inhibition of cell growth in a dose-dependent manner, with an EC₅₀ of about 25 nM. Addition of various concentrations (0.1 μM to 10 μM) of mutants partially inhibited the OxB-induced inhibition of cell growth. Indeed, addition of 10 μM of S18A/N20A or S18A/T27A peptides totally abolished the response induced by 0.1 nM OxB (FIGS. 3C and 3D), whereas both mutants partially antagonized the effect of 1 nM to 10 μM OxB. Moreover, Schild plots (Arunlakshana and Schild, 1959) derived from these experiments indicated that the regression line slope was about 0.8 for both analogs, suggesting that the mutants are not full competitive antagonists (data not shown). These results indicate that the double mutant S18A/N20A and S18A/T27A peptides, are partial agonists for apoptosis induction.

In order to determine the minimal sequence of OxB having a full biological activity, two deletion mutants were also synthesized (Table I). Deletion of the first five residues of OxB (OxB 6-28) did not alter the peptide ability to inhibit the binding of ¹²⁵I-OxA to OX1R as compared to native OxB (Table I). In contrast, deletion of the first six residues (OxB 7-28) induced a decrease in affinity for OX1R (Table I) by more than one log. OxB 6-28 induced apoptosis and IP3 production similarly as wt OxB (Table I) while OxB 7-28 strongly altered peptide-induced apoptosis and IP3 production (Table I). Taken together, these data suggest that the peptide must be at least 22-aminoacid long to induce these responses.

A 3D-Model of OX1R and Docking of the OxB C-Terminus.

BLAST analysis using OX1R sequence as a query against PDB database sequences, which include recent structural data of GPCRs, revealed that the best score was obtained with the neurotensin receptor, but only within a small region of the receptor (97 residues). The β2 adrenergic receptor associated to the Gs protein displayed the second best score and the homology covered the full sequence (329 residues), making it the best choice. Alignment between the β2 adrenergic receptor and the OX1R receptors was manually refined in order to align the two cysteine residues (C¹¹⁹ and C²⁰² in OX1R) present in TM III and extracellular loop (ECL), ECL 2, which are involved in a putative disulfide bridge in OX1R. Moreover, a manual modification of the alignment between OX1R and β2 adrenergic receptor was also performed to optimize the sequence alignment of TM III and TM VII. The resulting alignment indicated about 27% sequence identity and 47% sequence homology between these two receptors. It should be noted that the best sequence identities were observed in the TM domains (sequence identity range from 28 to 40%).

Based on this sequence alignment, a 3D-model of OX1R was constructed by homology modeling. The 3D-model of human OX1R with the best score was selected and subjected to energy minimization. The local root mean square deviation (rmsd) between the Cα of the TM core of OX1R and the β2 adrenergic receptor X-ray structure was evaluated to be 0.211 Å, indicating very close geometrical parameters for the two proteins. The ECL and intracellular loops (ICL) of OX1R were modeled on the basis of the β2 adrenergic receptor loops, except that ICL 3 was not taken into account in the 3D-model of OX1R because this loop was replaced in the β2 adrenergic receptor by the lysozyme T4 sequence to promote its crystallization (Rasmussen et al., 2011). The OX1R 3D-model exhibited one short helix in the intracellular C-terminal tail (L³¹⁷ to A³²⁷), as observed in the structure of β2 adrenergic receptor (Rasmussen et al., 2011). The C-terminus of OxB (sequence 20-28, named Ox20-28) was docked into OX1R by using HADDOCK software under constraints obtained from putative interactions between residues of the Ox20-28 peptide and residues present in OX1R. All calculated OX1R/Ox20-28 complexes were energy minimized in order to rearrange the side chains of the complexes. Among the various docking poses obtained, one 3D-model was selected on the basis of best HADDOCK score (−103.3±8.1), maximal buried surface area (1509 Å+64.6) and spatial orientation of the Ox20-28 C-terminus. Indeed, the model shows that the C-terminal M²⁸ residue of the Ox20-28 peptide is located well inside the binding pocket, whereas the N-terminal N²⁰ residue is oriented outside of the pocket.

Site-Directed Mutagenesis Analysis of the Receptor.

Based on the distance measured between the side-chain residues of the OX1R TM domains and those of the Ox20-28 peptide, we determined which residues of OX1R and Ox20-28 have side-chains within a distance <6 Å. All residues of OX1R fulfilling this condition were mutated to alanine by site-directed mutagenesis (Table II). It should be noted that the ECLs of OX1R were not taken into account in this experiment because sequence alignment between the β2 adrenergic receptor and OX1R revealed poor identity and homology in the ECL region. All mutants were functionally expressed in CHO—S cells (not shown) and tested for their abilities to bind ¹²⁵I-OxA and to induce apoptosis response, as above (Table II). Mutants I98A, C99A, P101A, V121A, Y211A, F220A, N318A, V319A, L320A, K321A and Y348A were able to bind ¹²⁵I-OxA and to induce apoptosis with an IC₅₀ in the nanomolar range, which is similar to the IC₅₀ determined for the wt-OX1R, e.g., IC₅₀mut/IC₅₀wt<10 and EC₅₀ₘᵤₜ/EC_{50wt}<10 (Table II). Of note, the substitution of Y³⁴⁸ by an alanine residue induced a slight inhibition of OX1R interaction with the peptide ligand and of apoptosis (Table II). In contrast, the Y124A, F340A, and T341A mutants displayed much lower affinity for ¹²⁵I-OxA than the wt-OX1R with a ratio IC₅₀mut/IC₅₀wt<25 (Table II). As shown in Table II, these mutants inhibited apoptosis, with a ratio EC₅₀mut/EC₅₀wt<25. It should be noted that the IC₅₀ for ¹²⁵I-OxA binding for these mutants was very similar to the EC₅₀ determined for the apoptosis response (Table II). However, substitution of K¹²⁰, P¹²³, H³⁴⁴ and W³⁴⁵ residues by an alanine residue had a strong impact on receptor affinity and the apoptotic response (Table II), indicating that these residues play a critical role in the interaction of OX1R with orexins. Indeed, the W345A mutant displayed a ratio IC₅₀mut/IC₅₀wt>60 and EC₅₀mut/EC₅₀wt>60 (Table II), whereas, the K120A, P123A, Y124A, F340A, T341A and H344A mutants displayed a ratio IC₅₀mut/IC₅₀wt>400 and EC₅₀mut/EC₅₀wt>400.

Discussion:

Several studies in recent years have shown that GPCRs represent new promising targets for the therapeutic treatment of various cancers (Lappano and Maggiolini, 2011). Among the large GPCR family, orexin receptors (OXR), a class A GPCR expressed in the hypothalamus, also display pro-apoptotic properties in cancer cell lines (Laburthe and Voisin, 2012). Our group demonstrated in the last few years that OX1R is not expressed in normal colon tissue, but is ectopically expressed in colon cancers where orexins bound to OX1R induce: 1) robust mitochondrial apoptosis (El Firar et al., 2009); and 2) significant inhibition of tumor growth in nude mice xenografted with cancer cell lines (Voisin et al., 2011). These effects are mediated via a novel mechanism involving: i) the presence of two ITIM (immunoreceptor tyrosine inhibitory motif) sequences in OX1R that are tyrosine phosphorylated upon receptor binding of orexins (Voisin et al., 2008); ii) the recruitment to tyrosine phosphorylated sites and activation of the tyrosine phosphatase, SHP-2, which is responsible for mitochondrial apoptosis involving cytochrome c release from mitochondria to cytosol and caspase-3 and caspase-7 activation (El Firar et al., 2009). In this context, the determination of the pharmacophore involved in the pro-apoptotic properties of the orexin peptide represents a key step for the design of new molecules with therapeutic interest. Although some studies regarding the pharmacophore determination of orexins to mobilize intracellular Ca²⁺ have been reported (Darker et al., 2001; Lang et al., 2004; German et al., 2013; Heifetz et al., 2013), no systematic evaluation of the pro-apoptotic function of every residue of OxB has been performed. In the present study, we have synthesized a total of 28 single alanine or leucine mutants of OxB and have analyzed their biological properties by binding assay, ability to stimulate intracellular IP3, and thereby Ca²⁺ release, and cellular apoptosis measurements in CHO—S cell clones expressing recombinant human OX1R. Our data provide critical new information on the key OxB amino acid residues that play a role in the pro-apoptotic function of the peptide.

Analysis of the 28 single alanine or leucine substitutions indicated that 12 residues in native OxB could not be changed without a significant decrease either in: i) the binding affinity for OX1R; ii) the ability to stimulate IP3 production or; iii) the ability to induce a pro-apoptotic response in transfected cells. These important residues were distributed mainly at the C-terminal end of the peptide chain, except for L¹¹ and L¹⁵, which are located in the peptide middle region. These results are in good agreement with previous observations indicating that the C-terminal end of OxB is crucial for both the binding to OX1R and the mobilization of intracellular Ca²⁺ (Lang et al., 2004). Moreover, the deletion of the first 5 residues of OxB had no impact on the ability of the truncated peptide to bind to OX1R, induce IP3 production and trigger cellular apoptosis. In contrast, the deletion of one more residue (OxB 7-28) strongly reduced the peptide activity, suggesting that the sequence 6-28 is essential for peptide function. Thus, we identified 7 residues, i.e., L¹¹, L¹⁵, A²², G²⁴, I²⁵, L²⁶ and M²⁸, as critical for binding to human OX1R and subsequent induction of cell apoptosis. It is quite interesting to note that all these residues in OxB are conserved in OxA, except for M²⁸, which is homolog to L³³ in OxA. Moreover, the analysis of the side-chain of these residues indicated mainly the presence of hydrophobic residues (L¹¹, L¹⁵, A²², G²⁴, and I²⁵ and L²⁶) and also the presence of one polar residue (M²⁸). Inversely, the substitution to alanine or leucine of the Q¹⁶, A¹⁷, S¹⁸, N²⁰ and T²⁷ residues had only a weak impact on binding affinity and Ca²⁺ mobilization, although it affected the induction of apoptosis. The structure determination of OxB by NMR had previously revealed the presence of two α-helices at position L⁷-G¹⁹ (Helix I) and A²³-M²⁸ (Helix II), connected by a flexible loop (Lee et al., 1999). Residues S¹⁸ and N²⁰ are located in the flexible loop, while T²⁷ is found in Helix II. Alanine substitution of S¹⁸, N²⁰ and T²⁷ strongly abolished peptide-induced apoptosis in CHO—S-OX1R, but the ability of these mutants to bind OX1R was not affected by alanine substitution of S¹⁸, and only slightly affected by alanine substitution of the N²⁰ and T²⁷ residues. These observations suggest that these three residues might play a key role in the induction of apoptosis mediated by orexins binding to their cognate receptors. The double substituted S18A/N20A and S18A/T27A peptides exhibited a slightly altered ability to interact with OX1R, indicating that substituting two of the three critical residues do not substantially alter affinity compared to single substitution of N²⁰ and T²⁷ residues. Moreover, these double substitutions totally abolished the ability of the two peptides to induce cellular apoptosis, suggesting that these peptides could be partial agonists/antagonists. Indeed, these two peptides partially antagonized the inhibition of cellular growth by OxB through a competitive mechanism. Thus, both the flexible loop and the C-terminal Helix II play a crucial role in the peptide main activity.

Sequence analysis of class A GPCRs revealed that there is a large diversity in the length and residue composition of the extracellular loops (ECLs). ECLs, and more particularly ECL2, which links TM4 and TM5, displayed different structures, including helices (for example, in some aminergic or adenosine receptors) or β sheets (for example, peptide-binding receptor) (Venkatakrishnan et al., 2013). A unique feature of the extracellular region of class A GPCRs is the presence of a disulfide bridge between a cysteine residue located in TM3 and a cysteine residue located in ECL2 (Fanelli and De Benedetti, 2011). The disulfide bridge has been shown to be involved in GPCR stability and activity (De Graaf et al., 2008). Indeed, the TM3-ECL2 disulfide bridge stabilized the extracellular side of TM3 close to the binding pocket, and limited the conformational change of this region during receptor activation (Preininger et al., 2013). The recent determination of the structure of the human β2 adrenergic receptor (Rasmussen et al., 2011), which shares 27% sequence identity with OX1R, allowed us to develop a homology 3D-model of OX1R that was subsequently used for ligand docking studies. The use of GPCR models in combination with site-directed mutagenesis represents an effective tool to study both ligand binding and functional properties. Therefore, the sequence alignment between the β2 adrenergic receptor and OX1R was manually refined in order to maintain the disulfide bridge between C¹¹⁹ (TM3) and C²⁰² (ECL2) of OX1R. The OX1R model exhibited an eighth helix (H8) at the top of the C-terminal sequence between residues L³¹⁷ to A³²⁷. The existence of H8 is frequently observed in class A GPCR, except for CXCR4, NTSR1 and PAR1 in which this region is unstructured (Venkatakrishnan et al., 2013). The structural and/or functional role of H8 in GPCRs remain conjectural, although some reports indicated a role in coupling and activation of G proteins (Rasmussen et al., 2011).

As mentioned above, the C-terminal end of OxB had a crucial role in the binding activity of the peptide, but also in the ability to induce a pro-apoptotic response. To understand how the C-terminal helix of OxB interacts with OX1R, we docked the C-terminal 20-28 fragment of OxB into the homology model of OXR1. Based on distance calculation (<6 Å) between residues of OX1R and OxB20-28, we substituted to alanine 18 residues of OX1R (see Table 2), showing that the substitution of 7 of these residues (K¹²⁰, P¹²³, Y¹²⁴, F³⁴⁰, T³⁴¹, H³⁴⁴ and W³⁴⁵), located in the TM2, TM3, TM6 and TM7 of OX1R, reduces the binding affinity of OxB to OX1R and inhibits the ability of OxB to induce apoptosis. Alanine substitution of K¹²⁰, P¹²³ and H³⁴⁴ resulted in a strong reduction of the ability of the receptor to bind OxB and to trigger an apoptotic response. These residues are located in TM3 and TM7 of OX1R, delineated the binding pocket. It should be noted that the substitution of H³⁴⁴ to alanine was previously shown to strongly reduce the ability of OX1R to mobilize intracellular calcium (Heifetz et al., 2013). Moreover, H³⁴⁴ was predicted to interact with L″ of OxB (Heifetz et al., 2013). K¹²⁰, located in TM3, is close to the C-terminal end of OxB, suggesting a putative interaction of this residue with H²¹ of OxB. In contrast, the substitution to alanine of P¹²³, which is located near K¹²⁰, could locally alter the structure of the top of TM3, modifying the orientation of the K¹²⁰ side chain. Previous report had revealed that substitution of residue Y³⁴⁸ did not modify the ability of OX1R to bind OxA and to mobilize intracellular calcium (Heifetz et al., 2013). In this study, the substitution of Y³⁴⁸ induced only a weak alteration of OX1R affinity and its ability to induce cellular apoptosis.

Conclusion:

The present “Ala-scan” study of OxB, associated with the development of a global 3D-model of OX1R and site-directed mutagenesis, demonstrated that the C-terminal end of OxB and 7 residues located in the transmembrane domains of OX1R are important for the induction of cellular apoptosis mediated by the OxB/OX1R interaction. As OX1R is aberrantly expressed in colon cancer and its activation by exogenous orexins result in robust apoptosis and strong inhibition of tumor development in preclinical animal models (Voisin et al., 2011), the design of full agonist peptide or non-peptide molecules represents a major challenge in new therapeutic approaches for the treatment of digestive cancers. The knowledge of the structure-function relationship of OxB and its receptor brought on by this study represents a key step in the development of such molecules.

TABLE I
Biological activity of OxB mutants in CHO-S cells stably expressing
human recombinant OX1R. IC₅₀ for inhibition of ¹²⁵I-OxA binding
and EC₅₀ for stimulation of IP3 production or apoptosis induction.
All parameters were determined in CHO-S-OX1R cells. Data are
mean ± S.E of at least three experiments performed in triplicate.

| | | Binding affinity | IP3 production | Apoptosis induction |
|---|---|---|---|---|
| OxB residues | Substitution | IC₅₀ (nM) | EC₅₀ (nM) | EC₅₀ (nM) |
| wt* | — | 31.2 ± 1.4 | 28.0 ± 5.8 | 19.6 ± 2.1 |
| R1 | Ala | 34.4 ± 2.7 | 51.0 ± 6.7 | 10.9 ± 0.9 |
| S2 | Ala | 51.0 ± 6.7 | 63.0 ± 49.6 | 30.3 ± 10.2 |
| G3 | Ala | 27.3 ± 2.2 | 14.0 ± 4.9 | 24.7 ± 4.1 |
| P4 | Ala | 34.3 ± 2.7 | 8.8 ± 5.6 | 21.1 ± 2.8 |
| P5 | Ala | 5.5 ± 0.8 | 10.8 ± 3.5 | 29.1 ± 7.0 |
| G6 | Ala | 46.7 ± 3.4 | 96.3 ± 18.2 | 33.9 ± 5.9 |
| L7 | Ala | 33.4 ± 8.3 | 32.2 ± 4.3 | 24.2 ± 4.6 |
| Q8 | Ala | 9.3 ± 0.7 | 8.1 ± 1.1 | 24.7 ± 4.1 |
| G9 | Ala | 46.1 ± 11.2 | 38.7 ± 11.4 | 50.2 ± 10.8 |
| R10 | Ala | 34.3 ± 2.7 | 14.2 ± 3.0 | 27.3 ± 2.2 |
| L11 | Ala | 300.1 ± 48.9 | 360.6 ± 136.0 | 1109.5 ± 475.5 |
| Q12 | Ala | 23.4 ± 1.7 | 6.7 ± 3.0 | 26.0 ± 7.1 |
| R13 | Ala | 32.2 ± 4.3 | 13.2 ± 2.6 | 20.3 ± 2.7 |
| L14 | Ala | 21.4 ± 5.5 | 14.0 ± 4.1 | 21.5 ± 2.4 |
| L15 | Ala | 375.1 ± 63.1 | 105.8 ± 26.4 | 3223.5 ± 1763.9 |
| Q16 | Ala | 29.4 ± 2.2 | 30.0 ± 4.9 | 368.6 ± 138.9 |
| A17 | Leu | 34.3 ± 2.7 | 31.6 ± 2.3 | 491.6 ± 258.3 |
| S18 | Ala | 20.7 ± 0.8 | 43.9 ± 6.2 | 5987.3 ± 2006.3 |
| G19 | Ala | 29.4 ± 2.2 | 12.5 ± 4.3 | 26.9 ± 7.0 |
| N20 | Ala | 157 ± 39 | 238.4 ± 79.9 | >10,000 |
| H21 | Ala | 30.5 ± 1.1 | 23.9 ± 3.9 | 106.0 ± 4.9 |
| A22 | Leu | 3491 ± 490 | >10,000 | 7504 ± 111.4 |
| A23 | Leu | 10.9 ± 0.9 | 18.4 ± 0.9 | 64.8 ± 12.0 |
| G24 | Ala | 3982.3 ± 820.3 | >10,000 | 7733.3 ± 2266.7 |
| I25 | Ala | 6674 ± 1663 | >10,000 | 3799.3 ± 1133.5 |
| L26 | Ala | 3563.7 ± 236.6 | >10,000 | >10,000 |
| T27 | Ala | 215.6 ± 70.4 | 142.4 ± 58.6 | >10,000 |
| M28 | Ala | 1263 ± 370.8 | 317.3 ± 93.2 | 7333.3 ± 2666.7 |
| OxB 6-28 | — | 47.4 ± 8.9 | 81.7 ± 24.9 | 75.1 ± 20.4 |
| OxB 7-28 | — | 388.0 ± 112.5 | 1513.7 ± 255.4 | 2183.0 ± 1133.6 |
| S18, N20 | Ala, Ala | 200.2 ± 58.8 | >10,000 | >10,000 |
| S18, T27 | Ala, Ala | 175.0 ± 24.5 | >10,000 | >10,000 |

*wt, wild type

TABLE II
Site-directed mutagenesis of OX1R. IC₅₀ for inhibition of ¹²⁵I-OxA
binding and EC₅₀ for apoptosis induction. All parameters were determined
in CHO-S cells stably expressing human recombinant OX1R. Data are
mean ± S.E of at least three experiments performed in triplicate.

| | | Binding affinity | IC₅₀mut/ | Apoptosis induction | EC₅₀mut/ |
|---|---|---|---|---|---|
| Mutants | Localization | IC₅₀ (nM) | IC₅₀wt | EC₅₀ (nM) | EC₅₀wt |
| wt* | — | 29.0 ± 5.2 | 1.0 | 28.3 ± 4.8 | 1.0 |
| I98A | TM2 | 41.4 ± 2.1 | 1.4 | 11.4 ± 0.7 | 0.4 |
| C99A | TM2 | 58.6 ± 12.0 | 2.0 | 31.2 ± 5.2 | 1.1 |
| P101A | TM2 | 58.1 ± 2.1 | 2.0 | 111.4 ± 10.8 | 3.9 |
| K120A | TM3 | >10,000 | >400 | >10,000 | >400 |
| V121A | TM3 | 52.1 ± 11.6 | 1.8 | 78.1 ± 12.4 | 2.8 |
| P123A | TM3 | >10,000 | >400 | >10,000 | >400 |
| Y124A | TM3 | 408.2 ± 73.5 | 14.0 | 295.0 ± 68.4 | 10.5 |
| Y211A | TM5 | 20.9 ± 0.9 | 2.3 | 35.5 ± 5.7 | 1.3 |
| F220A | TM5 | 21.4 ± 4.2 | 0.7 | 17.5 ± 3.8 | 0.6 |
| N318A | TM6 | 165.6 ± 44.7 | 5.7 | 151.1 ± 22.3 | 5.3 |
| V319A | TM6 | 24.8 ± 3.1 | 0.9 | 33.5 ± 6.2 | 1.2 |
| L320A | TM6 | 51.4 ± 17.8 | 1.8 | 17.5 ± 4.7 | 0.6 |
| K321A | TM6 | 132.5 ± 23.2 | 15.0 | 71.8 ± 16.2 | 2.5 |
| F340A | TM7 | 700 ± 74.9 | 24.0 | 517.5 ± 42.6 | 18.0 |
| T341A | TM7 | 358.1 ± 120.2 | 12.0 | 261.5 ± 58.2 | 9.0 |
| H344A | TM7 | >10,000 | >400 | >10,000 | >400 |
| W345A | TM7 | 2094.8 ± 88.5 | 72.0 | 1792.6 ± 74.5 | 64.0 |
| Y348A | TM7 | 37.2 ± 5.8 | 1.2 | 151.5 ± 28.9 | 5.4 |

*wt, wild type

### Example 2

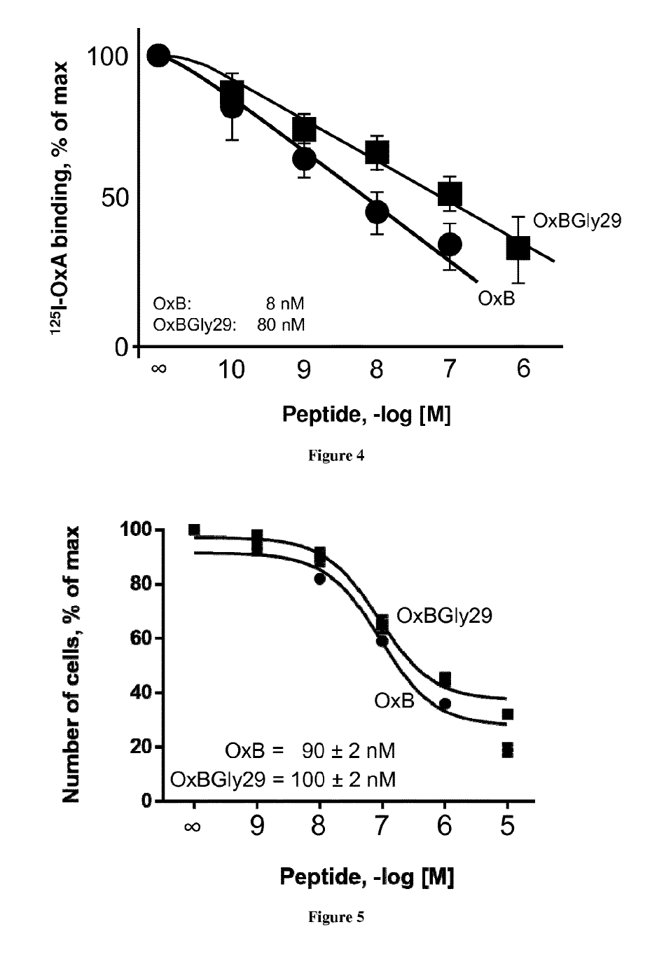

As mentioned by Lang et al. (Lang et al., 2004), the amidation of C-terminus of OxA and OxB peptides was crucial for receptor recognition. To overcome this requirement, we have developed a new peptide by addition of a glycine extra-residue at position 29 of OxB resulting of the OxBGly29 peptide which is no amidated at its C-terminus end. As shown in FIG. 4, the addition of a Gly29 residue slightly reduced the ability of OxBGly29 peptide to bind OX1R (IC50=80±2 nM) as compared to native peptide (IC50=8.0±0.4 nM). However, the addition of Gly residue at position 29 of OxB does not modified the ability of OxBGly29 to inhibit the cell growth of HEK-OX1R cells (EC50=100±2 nM) as compared to native peptide (EC50=90±2 nM) (FIG. 5). Taken together these results, addition of a Gly extra-residue at position 29 of OxB compensate well the absence of amidation at the C-terminal position of OxB peptide.

### REFERENCES

Throughout this application, various references describe the state of the art to which this invention pertains. The disclosures of these references are hereby incorporated by reference into the present disclosure.
Alexandre D, Hautot C, Mehio M, Jeandel L, Courel M, Voisin T et al. (2014) The orexin type 1 receptor is overexpressed in advanced prostate cancer with a neuroendocrine differentiation, and mediates apoptosis. Eur J Cancer. 50:2126-33.
Arunlakshana O, Schild H O (1959). Some quantitative uses of drug antagonists. Br J Pharmacol Chemother. 14:48-58.
Asahi S, Egashira S, Matsuda M, Iwaasa H, Kanatani A, Ohkubo M et al. (2003). Development of an orexin-2 receptor selective agonist, [Ala(11), D-Leu(15)]orexin-B. Bioorg Med Chem Lett.; 13:111-113.
Ceraudo E, Hierso R, Tan Y V, Murail S, Rouyer-Fessard C, Nicole P et al. (2012) Spatial proximity between the VPAC1 receptor and the amino terminus of agonist and antagonist peptides reveals distinct sites of interaction. FASEB J. 26:2060-71.
Ceraudo E, Murail S, Tan Y V, Lacapère J J, Neumann J M, Couvineau A, Laburthe M (2008) The vasoactive intestinal peptide (VIP) alpha-Helix up to C terminus interacts with the N-terminal ectodomain of the human VIP/Pituitary adenylate cyclase-activating peptide 1 receptor: photoaffinity, molecular modeling, and dynamics. Mol Endocrinol. 22:147-55.
Darker J G, Porter R A, Eggleston D S, Smart D, Brough S J, Sabido-David C et al. (2001) Structure-activity analysis of truncated orexin-A analogues at the orexin-1 receptor. Bioorg Med Chem Lett. 11:737-40.
De Graaf C, Foata N, Engkvist O, Rognan D (2008). Molecular modeling of the second extracellular loop of G-protein coupled receptors and its implication on structure-based virtual screening. Proteins 71:599-620
Dominguez C, Boelens R, Bonvin A M (2003). HADDOCK: a protein-protein docking approach based on biochemical or biophysical information. J Am Chem Soc. 125:1731-7.
El Firar A, Voisin T, Rouyer-Fessard C, Ostuni M A, Couvineau A, Laburthe M (2009). Discovery of a functional immunoreceptor tyrosine-based switch motif in a 7-transmembrane-spanning receptor: role in the orexin receptor OX1R-driven apoptosis. FASEB J. 23:4069-80.
Fanelli F, De Benedetti P G (2011). Update 1 of: computational modeling approaches to structure-function analysis of G protein-coupled receptors. Chem Rev. 111:PR438-535.
German N A, Decker A M, Gilmour B P, Thomas B F, Zhang Y (2013). Truncated Orexin Peptides: Structure-Activity Relationship Studies. ACS Med Chem Lett. 4:1224-1227.
Gotter A L, Webber A L, Coleman P J, Renger J J, Winrow C J (2012). International Union of Basic and Clinical Pharmacology. LXXXVI. Orexin receptor function, nomenclature and pharmacology. Pharmacol Rev. 64:389-420.
Heifetz A, Barker O, Morris G B, Law R J, Slack M, Biggin P C (2013). Toward an understanding of agonist binding to human Orexin-1 and Orexin-2 receptors with G-protein-coupled receptor modeling and site-directed mutagenesis. Biochemistry. 52:8246-60.
Laburthe M, Voisin T (2012). The orexin receptor OX(1)R in colon cancer: a promising therapeutic target and a new paradigm in G protein-coupled receptor signaling through ITIMs. Br J Pharmacol. 165:1678-87.
Laburthe M, Voisin T, El Firar A (2010). Orexins/hypocretins and orexin receptors in apoptosis: a mini-review. Acta Physiol (Oxf). 198:393-402.
Lang M, Soll R M, Durrenberger F, Dautzenberg F M, Beck-Sickinger A G (2004) Structure-activity studies of orexin a and orexin B at the human orexin 1 and orexin 2 receptors led to orexin 2 receptor selective and orexin 1 receptor preferring ligands. J Med Chem. 47:1153-1160.
Lappano R, Maggiolini M (2011). G protein-coupled receptors: novel targets for drug discovery in cancer. Nat Rev Drug Discov. 10:47-60.
Lee J H, Bang E, Chae K J, Kim J Y, Lee D W, Lee W (1999) Solution structure of a new hypothalamic neuropeptide, human hypocretin-2/orexin-B. Eur J Biochem. 266:831-9.
Le Guilloux V, Schmidtke P, Tuffery P (2009). Fpocket: an open source platform for ligand pocket detection. BMC Bioinformatics. 10:168.
Phillips J C, Braun R, Wang W, Gumbart J, Tajkhorshid E, Villa E et al. (2005) Scalable molecular dynamics with NAMD. J Comput Chem. 26:1781-802.
Preininger A M, Meiler J, Hamm H E (2013). Conformational flexibility and structural dynamics in GPCR-mediated G protein activation: a perspective. J Mol Biol. 425:2288-98.
Rasmussen S G, DeVree B T, Zou Y, Kruse A C, Chung K Y, Kobilka T S et al. (2011) Crystal structure of the β2 adrenergic receptor-Gs protein complex. Nature. 477:549-55.
Rouet-Benzineb P, Rouyer-Fessard C, Tarry A, Avondo V, Pouzet C, Yanagisawa M et al. (2004). Orexins acting at native OX(1) receptor in colon cancer and neuroblastoma cells or at recombinant OX(1) receptor suppress cell growth by inducing apoptosis. J Biol Chem. 279:45875-86.
Thompson M D, Xhaard H, Sakurai T, Rainero I, Kukkonen J P (2014). OX1 and OX2 orexin/hypocretin receptor pharmacogenetics. Front Neurosci. 57: 1-12.
Venkatakrishnan A J, Deupi X, Lebon G, Tate C G, Schertler G F, Babu M M (2013). Molecular signatures of G-protein-coupled receptors. Nature. 494:185-94.
Voisin T, El Firar A, Fasseu M, Rouyer-Fessard C, Descatoire V, Walker F et al. (2011). Aberrant expression of OX1 receptors for orexins in colon cancers and liver metastases: an openable gate to apoptosis. Cancer Res. 71:3341-51.
Voisin T, El Firar A, Rouyer-Fessard C, Gratio V, Laburthe M (2008). A hallmark of immunoreceptor, the tyrosine-based inhibitory motif ITIM, is present in the G protein-coupled receptor OX1R for orexins and drives apoptosis: a novel mechanism. FASEB J. 22:1993-2002.
Voisin T, Firar A E, Avondo V, Laburthe M (2006) Orexin-induced apoptosis: the key role of the seven-transmembrane domain orexin type 2 receptor. Endocrinology. 147:4977-84.
Xu T R, Yang Y, Ward R, Gao L, Liu Y (2013) Orexin receptors: multi-functional therapeutic targets for sleeping disorders, eating disorders, drug addiction, cancers and other physiological disorders. Cell Signal. 25:2413-23.

1. A polypeptide which comprises an amino acid sequence ranging from an amino acid residue at position 6 to an amino acid residue at position 28 in SEQ ID NO:2 wherein at least one amino acid residue at position 6, 7, 8, 9, 10, 12, 13, 14, 19, 21 or 23 is substituted and amino acid residues at positions 11; 15; 16; 17; 18; 20; 22; 24; 25; 26; 27; and 28 are not deleted or substituted.

2. The polypeptide of claim 1 wherein the amino acid residue at position 6, 7, 8, 9, 10, 12, 13, 14, 19, 21, or 23 is substituted by an alanine.

3. The polypeptide of claim 1 wherein a substitution is a conservative substitution.

4. The polypeptide of claim 1 which comprises 1, 4, 5, 6, 7, 8, 9, 10 or 11 substitutions in the amino acid sequence ranging from the amino acid residue at position 6 to the amino acid residue at position 28 in SEQ ID NO:2.

5. The polypeptide of claim 1 wherein a methionine residue at position 28 is amidated.

6. The polypeptide of claim 1 which is extended by at least one amino acid.

7. The polypeptide of claim 1 which is fused to a heterologous polypeptide to form a fusion protein.

8. The polypeptide of claim 1 which is fused to an immunoglobulin domain.

9. The polypeptide of claim 1 which is fused to an Fc portion to form an immunoadhesin.

10. A nucleic acid molecule which encodes for the polypeptide of claim 1.

11. The nucleic acid molecule of claim 10 which is included in a suitable vector

12. A host cell transformed with the nucleic acid molecule of claim 10.

13. (canceled)

14. A method of treating cancer in a subject in need thereof comprising administering to the subject a therapeutically effective amount of the polypeptide of claim 1.

15. The polypeptide of claim 6 wherein the at least one amino acid is glycine.
